(19) United States
(12) Patent Application Publication
(10) Pub. No.: US 2025/0381384 A1
(43) Pub. Date: Dec. 18, 2025

(54) Blood Control for a Catheter Insertion Device

(71) Applicant: C. R. Bard, Inc., Franklin Lakes, NJ (US)

(72) Inventors: Huy N. Tran, Riverton, UT (US); Mark A. Christensen, Salt Lake City, UT (US); Chad A. Hadley, North Salt Lake, UT (US); Jason R. Stats, Layton, UT (US)

(21) Appl. No.: 19/316,963

(22) Filed: Sep. 2, 2025

Related U.S. Application Data
(63) Continuation of application No. 18/238,298, filed on Aug. 25, 2023, now Pat. No. 12,403,294.
Application No. 18/238,298 is a division of application No. 16/696,844, filed on Nov. 26, 2019, now Pat. No. 11,759,618.
Application No. 16/696,844 is a division of application No. 15/702,537, filed on Sep. 12, 2017, now Pat. No. 10,493,262.
(60) Provisional application No. 62/393,531, filed on Sep. 12, 2016.

(51) Int. Cl. A61M 39/22; A61M 25/00; A61M 25/06; A61M 25/09; A61M 39/10; A61M 39/24

(52) U.S. Cl. CPC A61M 39/221; A61M 25/0097; A61M 25/065; A61M 39/24; A61M 25/0606; A61M 25/0631; A61M 25/0693; A61M 25/09041; A61M 39/10; A61M 2039/2426; A61M 2207/00

(57) ABSTRACT

A catheter placement tool includes a housing and a catheter assembly. The catheter assembly includes a fluid control component configured for controlling fluid flow through a hub of the catheter assembly during and after placement into the patient. The fluid control component is disposed within a cavity of the hub and is movable between a proximal position and a distal position. The fluid control component does not pierce a valve disposed in the hub when in the proximal position but pierces the valve when in the distal position. The fluid control component includes a conduit to enable fluid flow through an internal portion in the distal position. A plurality of longitudinal ribs can be disposed on an exterior surface of the fluid control component. The longitudinal ribs can provide fluid flow channels between the valve and an external portion of the fluid control component.

### CROSS-REFERENCE TO RELATED APPLICATIONS

This application is a continuation of U.S. patent application Ser. No. 18/238,298, filed Aug. 25, 2023, now U.S. Pat. No. 12,403,294, which is a division of U.S. patent application Ser. No. 16/696,844, filed Nov. 26, 2019, now U.S. Pat. No. 11,759,618, which is a division of U.S. patent application Ser. No. 15/702,537, filed Sep. 12, 2017, now U.S. Pat. No. 10,493,262, which claims the benefit of U.S. Provisional Patent Application No. 62/393,531, filed Sep. 12, 2016, and titled “BLOOD CONTROL FOR A CATHETER INSERTION DEVICE,” each of which is incorporated herein by reference in its entirety.

### BRIEF SUMMARY

Briefly summarized, embodiments of the present invention are directed to a tool for assisting with the placement into a patient of a catheter or other tubular medical device. In particular, a fluid control component configured for controlling fluid flow through the hub of the catheter assembly during and after placement into the patient is disclosed.

In one embodiment, the fluid control component comprises a body disposed within a cavity of the hub, the body being movable between a first position and a second position, wherein the body does not pierce a valve disposed in the hub when in the first position and wherein the body pierces the valve when in the second position. The body includes a conduit to enable fluid flow through an internal portion of the body when the body is in the second position, and a plurality of longitudinally extending ribs disposed on an exterior surface of the body. The ribs provide at least one fluid flow channel between the valve and an external portion of the body when the body is in the second position.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIGS. 1A and 1B are perspective views of a catheter insertion device according to one embodiment;

FIG. 2 is an exploded view of the catheter insertion device of FIGS. 1A and 1B;

FIG. 3 is a cross-sectional side view of a catheter according to one embodiment;

FIG. 4 is a perspective view of a blood control component of the catheter of FIG. 3;

FIGS. 5A and 5B show various views of the blood control component of FIG. 4;

FIGS. 6A-6C show various views of a blood control component for a catheter according to one embodiment;

FIGS. 7A-7C show various views of use of the catheter and blood control catheter of FIGS. 6A-6C;

FIGS. 8A-8F show various views of a needle hub of the catheter insertion device of FIGS. 1A and 1B;

FIG. 9 is a cross sectional view of a flash indicator of the catheter insertion device of FIGS. 1A and 1B;

FIG. 10 is a perspective view of a valve of the catheter insertion device of FIGS. 1A and 1B; and

FIG. 11 is an isolation view showing the blood control component piercing the valve of the catheter insertion device of FIGS. 1A and 1B.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used herein, including the claims, shall have the same meaning as the word “comprising.”

Embodiments of the present invention are generally directed to a tool for assisting with the placement into a patient of a catheter or other tubular medical device. For example, catheters of various lengths are typically placed into a body of a patient so as to establish access to the patient's vasculature and enable the infusion of medicaments or aspiration of body fluids. The catheter insertion tool to be described herein facilitates such catheter placement. Note that, while the discussion below focuses on the placement of catheters of a particular type and relatively short length, catheters of a variety of types, sizes, and lengths can be inserted via the present device, including peripheral IVs, intermediate or extended-dwell catheters, PICCs, central venous catheters, etc. In one embodiment, catheters having a length between about 1.25 inch and about 2.25 inches can be placed, though many other lengths are also possible.

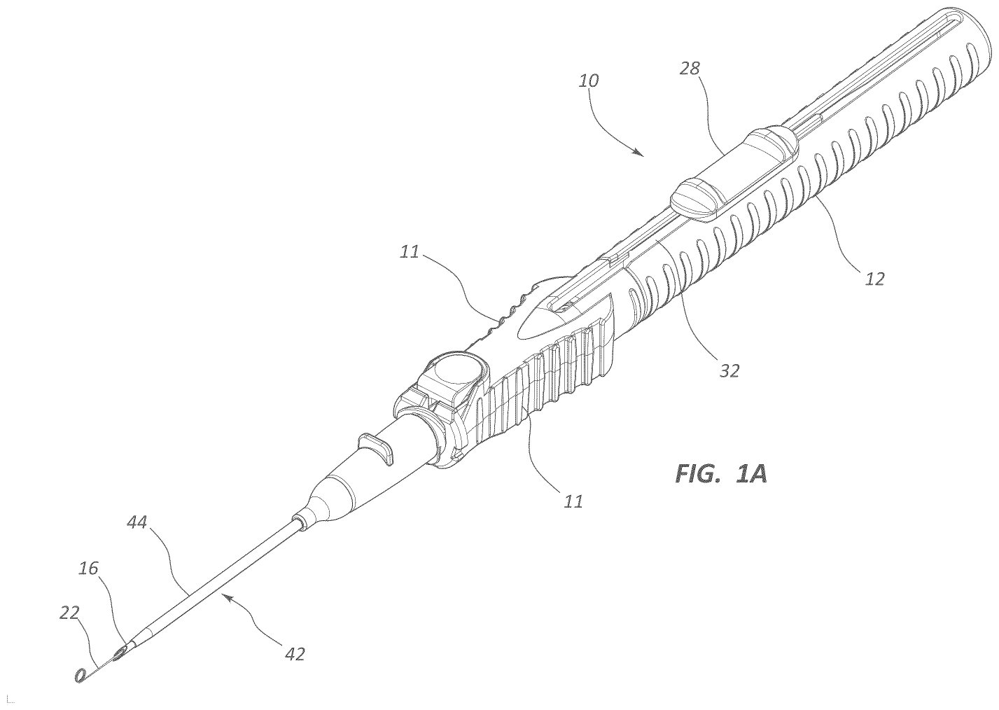

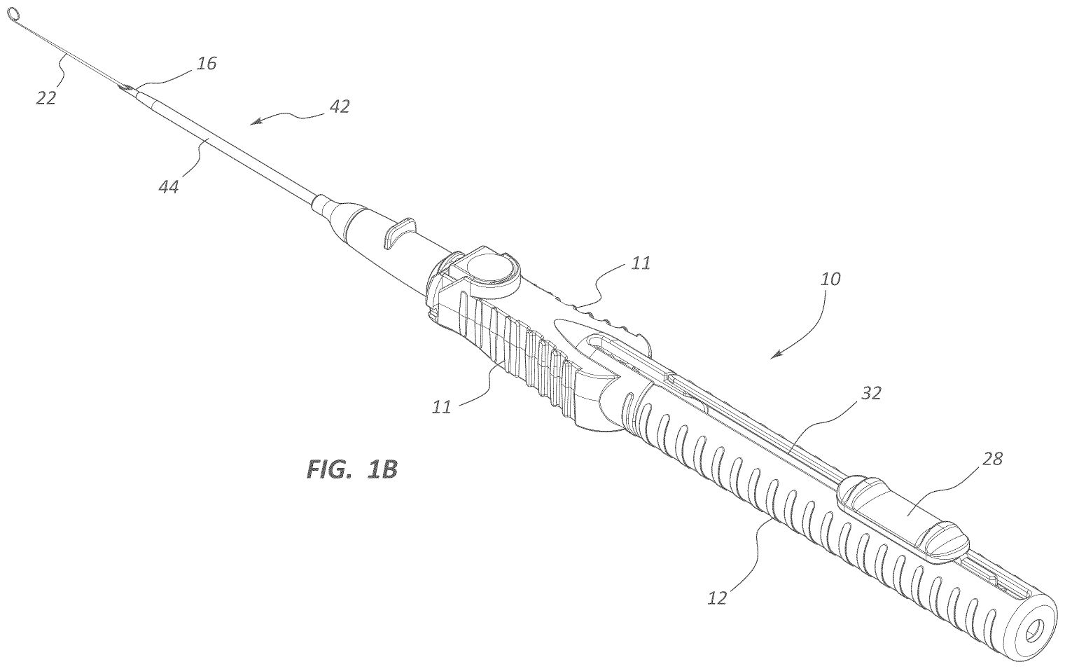

FIGS. 1A-2 depict various details regarding a catheter insertion tool (“insertion tool” or “insertion device”), generally depicted at 10, according to one embodiment. As shown, the insertion tool 10 includes a housing 12 that may itself include a proximal housing portion 12A and a distal housing portion 12B. The housing 12 further includes an open distal end, and can include a flat bottom to enable the insertion device 10 to lie flat on a surface without tipping. In another embodiment, the housing is integrally formed. In yet another embodiment, a top housing portion and a bottom housing portion can be employed, or more than two portions can be used. In the present embodiment, the housing composed of a thermoplastic such as polycarbonate and is translucent, though other configurations are contemplated. The housing 12 defines grip surfaces 13 on either side of the housing, as seen in FIGS. 1A and 1B, to enable grasping of the insertion device 10 by the user.

A needle hub 14 supporting a hollow needle 16 (which together form part of a needle assembly, in one embodiment) is included with the housing 12. In the present embodiment, the needle hub 14 is secured within the housing 12 within a cavity 13 defined by the housing, but in another embodiment it can be integrally formed with the housing.

As will be described in detail further below, the needle hub 14 includes a slot for receiving a portion of the needle 16 and a quantity of adhesive, such as liquid or UV-cure adhesive for example, in order to fix the needle in place in the needle hub. The needle 16 extends distally from the needle hub 14 so as to extend past the distal end of the distal housing portion 12B and terminates at a distal end 16B thereof. A notch 18 is defined through the wall of the needle 16 proximate the distal end thereof. The notch 18 enables flashback of blood to exit the lumen defined by the hollow needle 16 once access to the patient's vasculature is achieved during catheter insertion procedures. Thus, blood exiting the notch 18 can be viewed by a clinician to confirm proper needle placement in the vasculature, as will be explained further below.

A catheter 42 including a catheter tube 44 is removably disposed on the portion of the needle 16 residing external to the housing 12 such that the needle occupies a lumen of the catheter defined by a catheter tube. The catheter tube 44 extends distally from a hub 46 of the catheter 42, which hub is initially disposed adjacent the open distal end of the distal housing portion 12B, as shown in FIGS. 1A and 1B.

The insertion device 10 further includes a guidewire advancement assembly 20 for advancing a guidewire 22 through the needle 16 and into the vasculature of the patient once vessel access by the needle has been achieved. The guidewire 22 (FIGS. 1A-2) is pre-disposed within the lumen of the needle 16. The guidewire advancement assembly 20 includes a guidewire lever 24 that selectively advances the guidewire 22 in a distal direction during use of the insertion device 10 such that the distal portion of the guidewire extends beyond the distal end 16B of the needle 16. In the present embodiment, a finger pad 28 of the guidewire lever 24 is slidably disposed on the housing 12 via a slot 32 to enable a thumb and/or finger(s) of the user to selectively advance the guidewire 22 distally past the distal end 16B of the needle 16. Of course, other engagement schemes to translate user input to guidewire movement could also be employed. In one embodiment, the guidewire 22 can include a guidewire support tube to provide additional stiffness to the guidewire and facilitate its distal advancement described above. In yet another embodiment, a proximal end of the guidewire can be attached at an anchor point on an interior portion of the housing 12 (or other fixed portion of the insertion device 10) and looped about a proximal portion of the guidewire lever 24 in a roughly U-shaped configuration such that the distal end of the guidewire extends two units of distance distally past the distal end 16B of the needle 16 for every one unit of distance of movement of the finger pad 28. These and other modifications are therefore contemplated.

The majority length of the guidewire in one embodiment includes a metal alloy of nickel and titanium commonly referred to as nitinol, though other suitable guidewire materials can also be employed.

FIGS. 1A and 1B show that the catheter 42 is removably attached to the insertion device 10 such that the catheter tube 44 thereof is disposed over the portion of the needle 16 that extends distal to the housing 12 such that the catheter resides external to the insertion device housing. The catheter 42 in the present embodiment is kept in place against the open distal end of the housing via a friction fit with one or more features disposed on the housing distal end. A tab 48 is included on the catheter hub 46 for assisting with manual distal extension of the catheter 42 by a user during deployment thereof.

Note that in one embodiment the outer diameters (and/or other areas) of the needle 16 and the catheter tube 44 are lubricated with silicone or other suitable lubricant to enhance sliding of the catheter tube with respect to the needle and for aiding in the insertion of the catheter into the body of the patient.

The insertion device 10 includes a retraction system configured to selectively retract the needle 16 into the housing 12. In detail, a spring element, such as a coil spring 50, is disposed between a distal end of the inner cavity 13 of the housing 12 and a ridge 144 disposed at a proximal end of the needle hub 14. The spring 50 is disposed about the needle hub 14, and the needle hub is proximally slidable within the cavity of the housing 12. The needle hub 14 is kept in a distal position within the cavity of the housing 12, with the spring maintained in a compressed configuration, by a retraction button 52 disposed near the distal end of the housing 12. Manual depression of the retraction button 52 releases engagement of the retraction button with the needle hub 14, which in turn enables the spring 50 to expand, causing the needle hub to move proximally within the cavity of the housing 12. This in turn retracts the needle 16 so that the distal end 16B thereof is retracted into the housing 12 and protected from inadvertent contact by a user. Note that other needle safety configurations can also be employed.

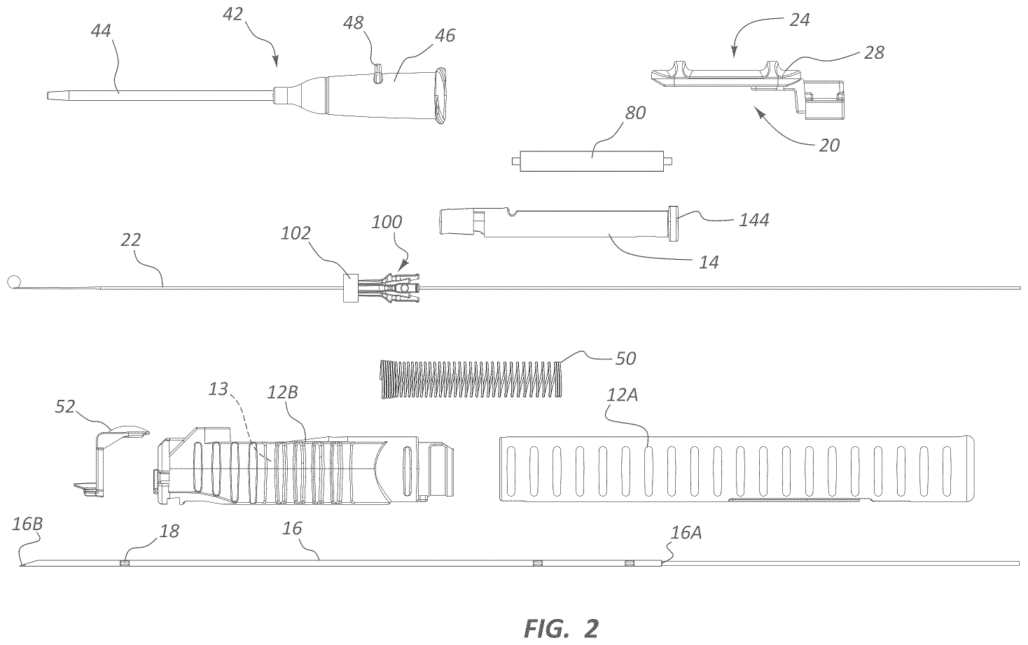

Use of the insertion device 10 in placing the catheter 42 in the vasculature of a patient is described here. A user grasping the insertion device 10 first guides the distal portion of the needle 16 through the skin at a suitable insertion site and accesses a subcutaneous vessel. After needle access to the vessel is confirmed, the guidewire advancement assembly 20 is actuated, wherein the finger pad 28 (disposed in the slot 32 defined in the housing 12) is advanced by the finger of the user to distally advance the guidewire 22 (FIG. 3), initially disposed within the hollow needle 16. Note that the guidewire 22 is distally advanced by the guidewire lever 24, which is operably attached to the slidable finger pad 28.

Distal advancement of the guidewire 22 continues until the finger pad 28 has been distally slid a predetermined distance, resulting in a predetermined length of the guidewire 22 extending past the distal end of the needle 16, as shown in FIGS. 1A and 1B. This places the distal portion of the guidewire 22 within the vessel.

Once the guidewire lever 24 has been fully distally extended via sliding of the finger pad 28, which in turn has extended the guidewire 22 past the distal end 16B of the needle 16, manual distal advancement of the catheter 42 is performed, using the tab 48 of the catheter hub 46, which causes the catheter tube 44 to slide over distal portions of the needle 16 and guidewire 22 and into the patient's vasculature via the insertion site. In light of this, it is appreciated that the finger pad 28 acts as a first member used to advance the guidewire 22, whereas manual advancement is employed to advance the catheter 42, in the present embodiment. In another embodiment, it is appreciated that the finger pad 28 can be employed to also distally deploy the catheter 42 at least a partial distance into the vessel.

The catheter 42 is distally advanced until it is suitably disposed within the vessel of the patient. The retraction button 52 on the housing 12 is then manually depressed by the user, which causes the spring 50 to decompress and retract the needle hub 14, which in turn causes the distal end 16B of the needle 16 to be retracted within the housing 12 and preventing its re-emergence, thus protecting the user from accidental needle sticks. Thus, this serves as one example of a needle safety component, according to the present embodiment; others are possible. The catheter 42 is physically separated from the housing 12 at this time. Now in place within the patient, the catheter 42 can be prepared for use and dressed, per standard procedures. Then insertion device 10 can be discarded.

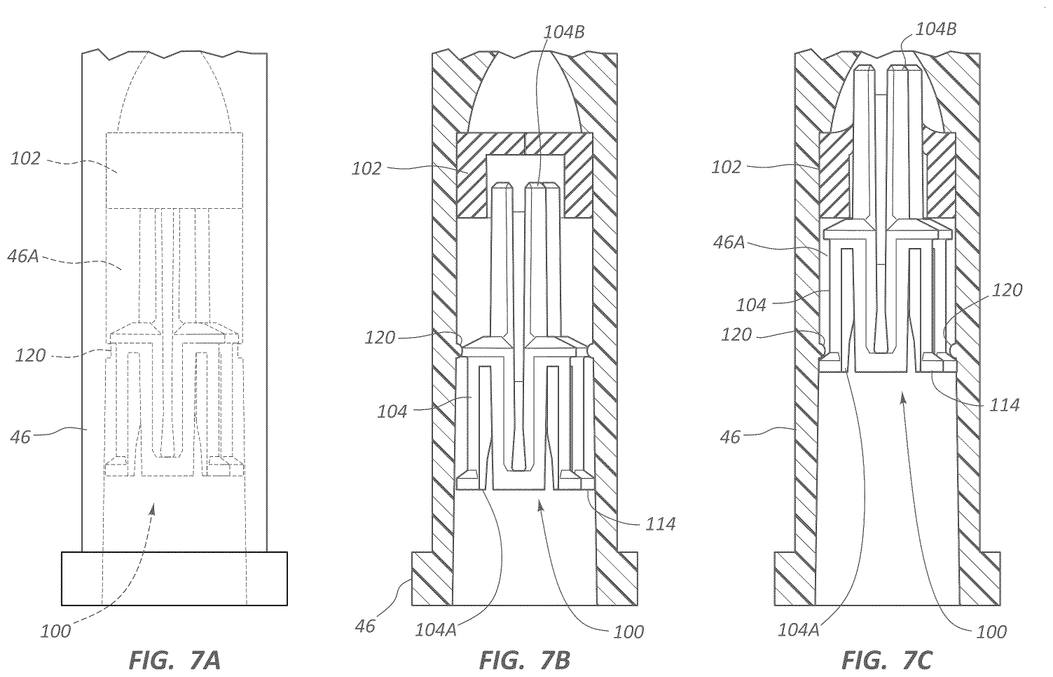

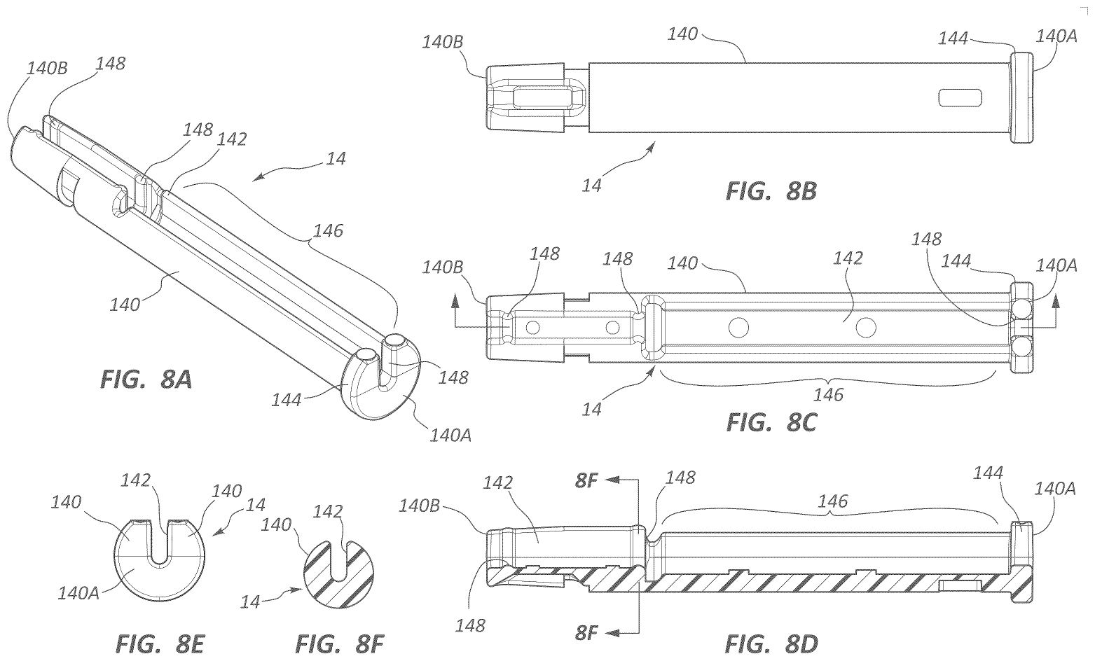

In additional detail, FIG. 2 shows a continuous blood flash indicator 80 that can be used with the insertion device 10 according to one embodiment. The flash indicator 80 is employed to indicate the presence of blood in the lumen of the needle 16 during use of the device 10, thus assuring that proper access has been made by the needle into a vein or other desired blood-carrying vessel. As shown in FIG. 9, the flash indicator 80 includes a translucent chamber 82 that is generally cylindrical in shape, sealed at either end, and disposed about a portion of the needle 16 such that the needle protrudes out from either sealed end. In the present embodiment the chamber 82 of the flash indicator 80 is disposed in the slot 142 (FIGS. 8A-8F) of the needle hub 14 within the housing 12, though other locations along the needle are also possible.

Two notches—a first notch 83 and a second notch 84—are defined in the needle 16 so as to provide fluid communication between the lumen of the needle and the interior of the flash indicator chamber. The notches 83, 84 replace the notch 18 (FIG. 2) in one embodiment, and are included in addition to the notch 18, in another embodiment. It is appreciated that, in one embodiment, blood passage through the notch 18 serves as an initial indicator that the distal end 16B of the needle has entered the vein, while the embodiment shown here serves as an additional indicator to verify that the needle distal end remains in the vein after initial access. Further detail regarding the flash indicator 80 can be found in U.S. Publication No. 2016/0331938, published Nov. 17, 2016, and entitled “Catheter Placement Device Including an Extensible Needle Safety Component,” which is incorporated herein by reference in its entirety.

In the present embodiment, the guidewire 22 passes through the lumen of the needle 16 so as to extend through the flash indicator 80. The first notch 83 is disposed distal to the second notch 84 toward the distal end of the chamber 82.

When vessel access is achieved by the distal end 16B of the needle 16, blood travels proximally up the lumen of the needle, between the inner surface of the needle and the outer surface of the guidewire 22, disposed in the needle lumen (FIG. 9). Upon reaching the relatively more distal first notch 83 defined in the needle 16, a portion of the blood will pass through the first notch and enter the chamber 82. As the blood fills the translucent chamber 82, a user can observe the chamber through the translucent housing 12 of the insertion device 10 and view the blood therein, thus confirming that the vessel access has been achieved. In another embodiment, the housing 12 can be configured such that direct viewing of the chamber 82 is possible, e.g., with no intervening structure interposed between the chamber and the user.

The second notch 84 is employed to provide an exit point for air in the chamber 82 to equalize air pressure and enable the blood to continue entering the chamber via the first notch 83. It is noted that the spacing between the inner surface of the needle 16 and the outer surface of the guidewire 22 along section 85 is such that air but not blood can pass therebetween, thus enabling air pressure equalization in the chamber without blood passage through the second notch 84. In this way, the flash indicator 80 is a continuous indicator, enabling a continuous flow of blood into the chamber 82 while the needle distal end 16B is disposed within the blood-carrying vessel.

Note that the catheter insertion device 10 can include more than one flash indicator. In one embodiment and as mentioned above, for instance, the blood flash indicator 80 can be included, along with another flash indicator, such as the notch 18, which enables blood present in the lumen of the needle 16 to proceed proximally up the space between the outer surface of the needle and the inner surface of the catheter 42.

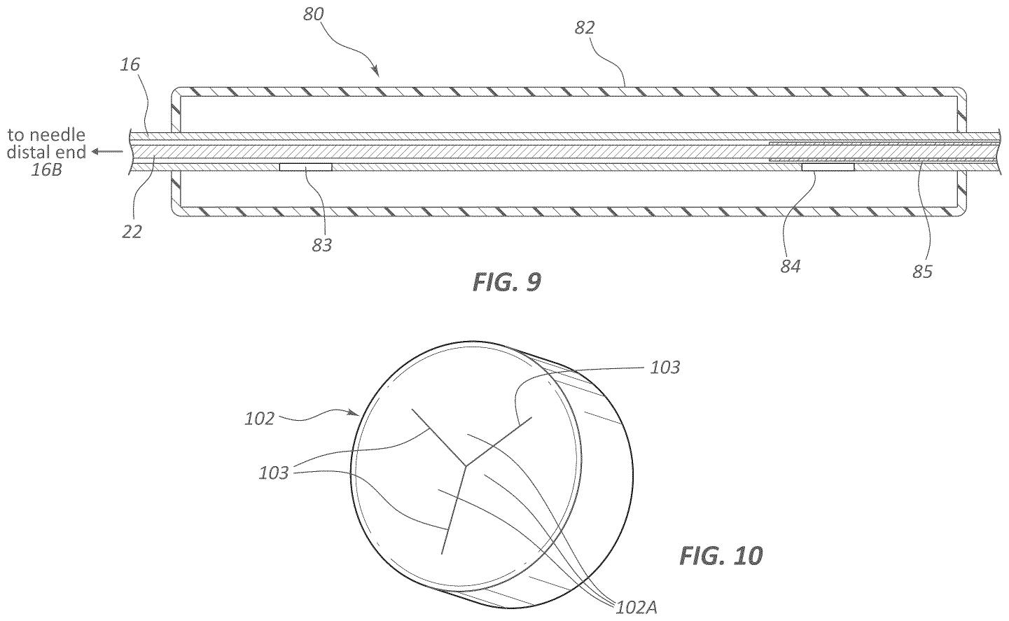

FIG. 3 depicts various details of a blood control component 100 included with the catheter 42, in accordance with one embodiment. As shown, the blood component 100 is slidably disposed within a cavity 46A of the catheter hub 46 and is configured to selectively enable fluid flow through the catheter 42 in concert with a valve 102, also disposed within the catheter hub cavity. The valve 102 in the present embodiment is a tricuspid valve including three leaflets 102A defined by a plurality of slits 103 as seen in FIG. 10, though other valve types may also be employed.

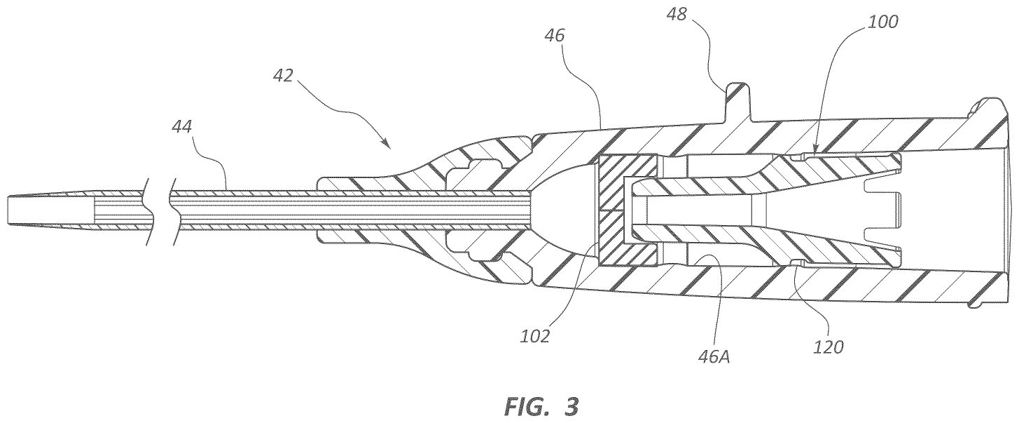

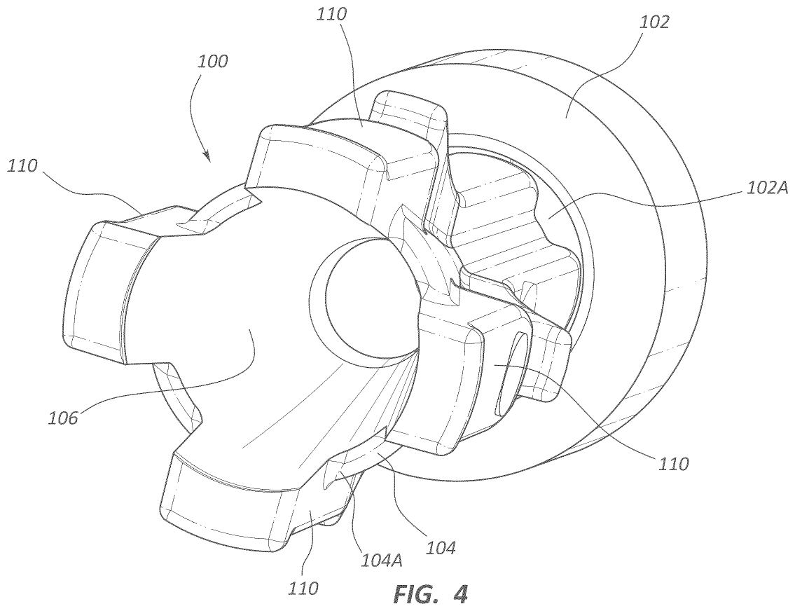

FIGS. 4-5B depict various details of the blood control component 100, including an elongate body 104 extending between a proximal end 104A and a distal end 104B and defining a central conduit 106 through which fluids can flow. A plurality of ribs 110 is disposed on an outer surface of the body 104 such that the ribs longitudinally extend from proximally past the proximal end 104A of the body to the distal end 104B thereof. Each rib 110 radially extends from the body 104 to define a contoured profile along the longitudinal length thereof. The body 104 and ribs 110 contribute to generally define a conical shape to the blood control component 100. Deviations from the conical shape are also possible in other embodiments.

Each rib 110 further defines a notch 112 intermediately positioned along the longitudinal length of the rib, as well as a protrusion 114 at the proximal end of the rib. As seen in FIG. 3, the notch 112 of each rib 110 receives a portion of an annular ridge 120 defined on an inner surface of the catheter hub cavity 46A to keep the blood control component 100 in place within the cavity before actuation. Correspondingly, the protrusions 114 of each rib 110 engage with the annular ridge 120 when the blood control component 100 is actuated so as to prevent further distal movement thereof past its intended length of travel. The body 104 defines a channel 126 between adjacent ribs 110, thus providing four fluid flow channels in the illustrated embodiment. Note that in one embodiment one or more ribs 110 can be offset along the longitudinal length thereof such that a proximal portion of the rib including the protrusion 114 is not longitudinally aligned with (as in FIGS. 5A and 5B), but rather circumferentially offset from, a more distal portion of the rib.

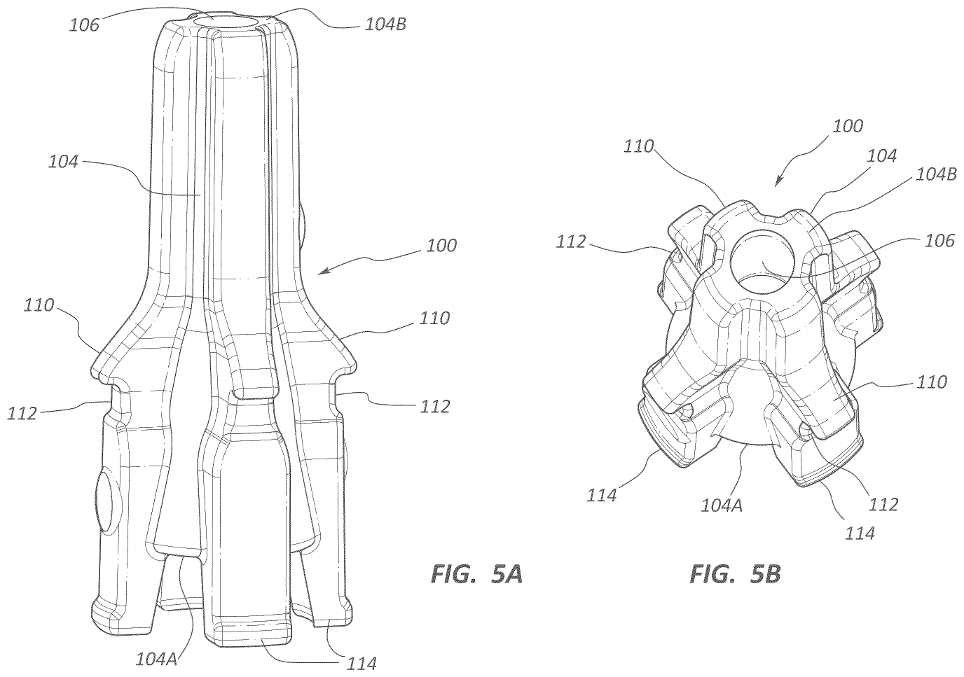

FIGS. 6A-6C depict details of the blood control component 100 according to another embodiment, wherein the body 104 defines a plurality of channels 126 disposed about the conduit 106. An intermediate, annular shoulder 128 is also defined by the body 104.

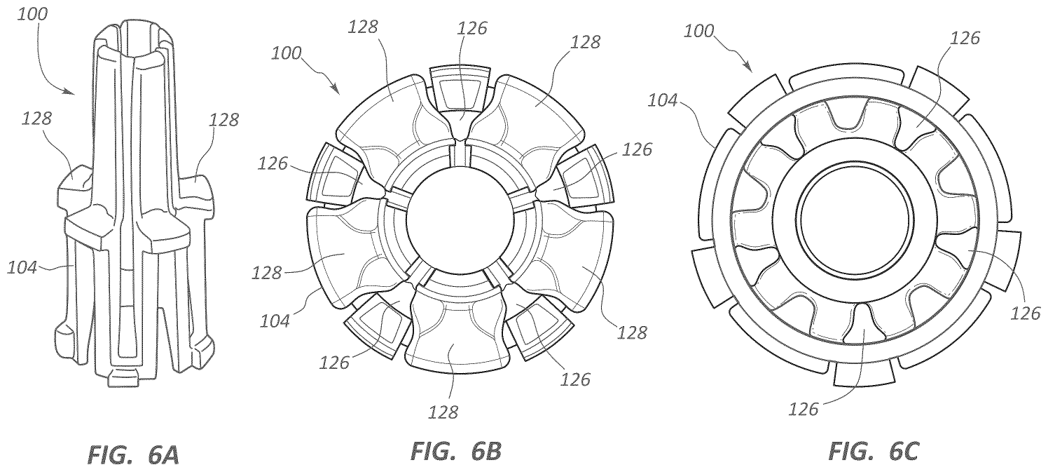

FIGS. 7A-7C depict various stages of operation of the blood control component 100 of FIGS. 6A-6C, though the principles described here also apply to the embodiment shown in FIGS. 4-5B as well. In particular, FIGS. 7A and 7B show the blood control component 100 in a relatively proximal position, also referred to herein as an un-actuated state, wherein the annular ridge 120 is received within the notches 112 (below the shoulders 128) of each rib 110 of the blood control component. In this position, the distal end 104B of the body 104 does not protrude through the valve 102 that is positioned distal to the blood control component and thus no fluid is able to pass through the catheter 42, as desired. The valve 102 in the closed position thus prevents blood leakage through the catheter 42, such as when the catheter has been placed within the patient but no external connection has been made to the catheter hub 46.

In contrast, FIG. 7C shows the blood control component 100 in a relatively distal position, also referred to herein as an actuated state, wherein the blood control component has been distally advanced (such as by insertion of a male luer connector into the catheter hub 46) such that the distal end 104B thereof has penetrated through the leaflets 102A of the valve 102, thus providing a fluid path through the valve via the conduit 106 of the blood control component. Further distal advancement of the blood control component 100 is prevented by engagement of the protrusions 114 against the annular ridge 120. As mentioned, the distal movement of the blood control component 100 is caused by the insertion into the catheter hub cavity 46A by a luer connector or other apparatus that can be operably connected to the catheter hub 46.

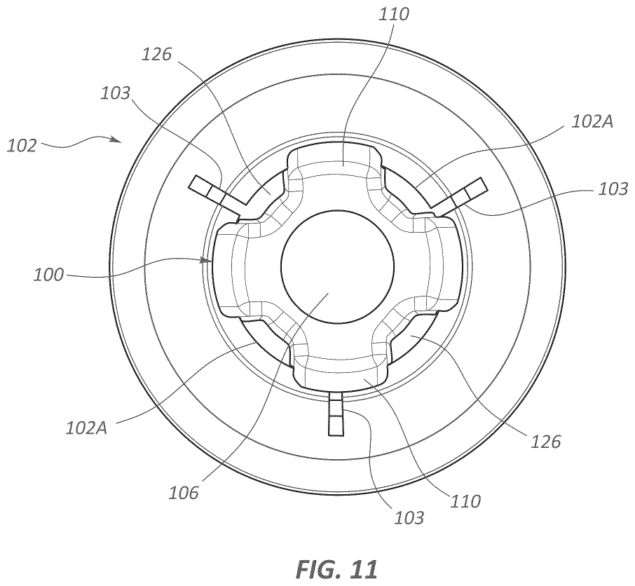

In accordance with the present embodiment, the blood control component 100 is configured to eliminate an entrapment zone between the blood control component and the valve 102 after the blood control component has pierced the valve in its actuated state. Specifically, and with respect to the embodiment shown in FIGS. 4-5B, the ribs 110 cause additional deformation of the leaflets 102A of the valve 102 when the blood control component pierces the valve, as seen in FIG. 11. This in turn prevents partial sealing of the leaflets 102A to the exterior surface of the blood control component body 104, thus providing spacing therebetween and additional fluid flow paths via the channels 126 between the exterior surface of the blood control component body 104 and the valve leaflets. Thus, fluid is able to flow through the catheter hub cavity 46A not only internal to the blood control component body 104 via the conduit 106 but also external to the blood control component body via the channels 126, which are made patent by the interaction of the ribs 110 with the valve leaflets 104A. This fluid flow external to the blood control component 100 assists in moving fluid through the entirety of the hub cavity 46A, thus desirably preventing fluid flow stagnation in the region between the blood control component 100 and the valve 102.

Note that in the present embodiment an outer termination point of each slit 103 that form the leaflets 102A defines a staggered termination point, as seen in FIG. 11. Note also that the ribs described herein are but one example of one or more extended surfaces that can be included with the blood control component to enable additional fluid flow channels to be defined on an outer surface of the blood control component to enable fluid flow about the exterior of the blood control component when the blood control component pierces the valve. Examples of other extended surfaces include bumps, annular surfaces, fins, etc. These and other embodiments are therefore contemplated.

The blood control component 100 of FIGS. 6A-6C operates similarly to that described immediately above in connection with FIGS. 4-5B, wherein the channels 126 provide fluid flow in addition to the conduit 106 so as to prevent fluid flow stagnation between the blood control component 100 and the valve 102.

FIGS. 8A-8F depict various details regarding the aforementioned needle hub 14 of the insertion device 10, which includes an elongate body 140 extending between a proximal end 140A and a distal end 140B. A slot 142 extends longitudinally along the length of the body 140 and is sized for receiving a portion of the length of the needle 16 therein. As mentioned, the ridge 144 is included on the proximal end 140A of the needle hub and provides a surface against which the spring 50 can act to retract the needle hub and attached needle 16 into the cavity of the housing 12. The slot 142 defines a volume 146 within which the above-described flash indicator 80 can be received.

Note that the slot 142 is configured so that differing sizes of needle can be received and affixed therein. To that end, the slot 142 includes three shoulders 148 to support the needle 16 within the slot 142. Note that the proximal edge of each of the shoulders 148 is relatively abrupt in shape so as to prevent spillage of a liquid epoxy adhesive that is placed in the slot 142 proximate the shoulders to secure the needle 16 within the slot.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A catheter placement tool, comprising:
a housing, comprising:
a needle including a distal notch in a sidewall of the needle, the distal notch proximate a distal end of the needle, and a pair of proximal notches in the sidewall of the needle proximate a proximal end of the needle, wherein the distal notch and the pair of proximal notches are in fluid communication with a lumen of the needle;
a flash chamber enclosing the pair of proximal notches; and
a needle hub including a slot for receiving a proximal portion of the needle and the flash chamber; and
a catheter assembly, comprising:
a catheter including a catheter hub coupled to a proximal end of a catheter tube, the catheter hub including a valve and an annular ridge defined on an inner surface of the catheter hub proximal of the valve; and
a fluid control component disposed in the catheter hub, the fluid control component movable between a proximal position and a distal position, wherein:
a portion of the fluid control component penetrates the valve in the distal position, and
the fluid control component includes a protrusion at a proximal end configured to engage the annular ridge in the distal position.

2. The catheter placement tool according to claim 1, wherein the valve of the catheter includes a tricuspid valve including three slits.

3. The catheter placement tool according to claim 2, wherein an outer termination point of each slit is a staggered termination point.

4. The catheter placement tool according to claim 1, wherein the fluid control component includes a plurality of longitudinal ribs spaced apart on an exterior surface, each of the plurality of longitudinal ribs extending from the proximal end of the fluid control component to a distal end of the fluid control component, each of the plurality of longitudinal ribs comprising a notch at an intermediate portion between the proximal end and the distal end, the notch configured to engage the annular ridge in the proximal position.

5. The catheter placement tool according to claim 4, wherein each of the plurality of longitudinal ribs includes a shoulder adjacent to the notch.

6. The catheter placement tool according to claim 4, wherein the plurality of longitudinal ribs comprise four equally spaced longitudinal ribs.

7. The catheter placement tool according to claim 4, wherein each of the plurality of longitudinal ribs extend proximal of a proximal opening of the fluid control component.

8. The catheter placement tool according to claim 4, wherein the fluid control component further comprises a fluid flow channel between each of the plurality of longitudinal ribs.

9. The catheter placement tool according to claim 1, wherein the flash chamber is formed from a translucent material.

10. The catheter placement tool according to claim 1, wherein the slot of the needle hub includes a plurality of shoulders configured to support the needle, wherein a proximal shoulder includes a proximal edge configured to prevent the flow of a liquid adhesive out of the slot when the liquid adhesive is used to secure the needle in the slot.

11. The catheter placement tool according to claim 1, wherein the needle hub includes a radially extending ridge disposed proximate a proximal end of the needle hub, the radially extending ridge configured to support a spring of the insertion device.

12. The catheter placement tool according to claim 11, further comprising a retraction system configured to selectively retract the needle into the housing, the retraction system comprising the spring and a retraction button in releasable engagement with the needle hub.

13. The catheter placement tool according to claim 1, further comprising a guidewire advancement assembly including a guidewire pre-disposed in the lumen of the needle.

14. The catheter placement tool according to claim 13, wherein the guidewire advancement assembly further includes a finger pad coupled to the guidewire, wherein the guidewire is designed for distal advancement upon distal advancement of the finger pad.

15. The catheter placement tool according to claim 1, wherein the fluid control component has a generally conical shape.
